(19) United States
(12) Patent Application Publication
(10) Pub. No.: US 2022/0339363 A1
(43) Pub. Date: Oct. 27, 2022

(54) Injection Device Comprising a Needle Shield

(71) Applicant: Becton Dickinson France, Le Pont de Claix (FR)

(72) Inventors: Benoit Zeller, Annecy le Vieux (FR); Gilles Bernede, Arbusigny (FR)

(21) Appl. No.: 17/764,370

(22) PCT Filed: Oct. 14, 2020
(86) PCT No.: PCT/EP2020/078926

§ 371 Date: Mar. 28, 2022

(30) Foreign Application Priority Data
Oct. 23, 2019 (EP) 19315129.7

(51) Int. Cl. A61M 5/315; A61M 5/31

(52) U.S. Cl. CPC A61M 5/3158; A61M 5/31; A61M 2005/3247

(57) ABSTRACT

Provided is an injection device, comprising a syringe comprising a needle, a barrel, a stopper in sliding engagement inside the barrel, a plunger unit moveable with regard to the barrel, a case accommodating the barrel and provided with elastic means and an operating stop, and a needle shield adapted to be moveable under the force of the elastic means from an injection position to a safety position, and provided with a shield stop, wherein the operating stop is configured to prevent the needle shield to move to the safety position by abutting against the shield stop, wherein the plunger unit comprises a triggering portion configured to deflect the flexible leg of the needle shield when the plunger unit has reached a triggering position during its injection movement.

### CROSS-REFERENCE TO RELATED APPLICATIONS

This application is the United States national phase of International Application No. PCT/EP2020/078926 filed Oct. 14, 2020, and claims priority to European Patent Application No. 19315129.7 filed Oct. 23, 2019, the disclosures of which are hereby incorporated by reference in their entirety.

### BACKGROUND OF THE INVENTION

### Technical Field

The present invention relates to an injection device, and in particular, to a medical injection device to be used by a user to inject a substance into a body and comprising a needle shield arranged to cover the needle when the injection device is not used.

### Description of Related Art

An injection device provided with a needle shield is known. Such a needle shield may be in a deployed or extended position before use of the injection device to avoid needle phobia before injection, move to a retracted position during regular pricking/injection, and then move back to another deployed position in which it is locked after use, to allow safe handling and/or discarding of the injection device without any risk of injuries.

However, locking the needle shield in the deployed position after movement from the retracted position to the deployed position might be problematic. Indeed, in case the user needs to change the injection site, the needle shield is locked in the deployed position and does not permit any pricking or injection anymore, thereby preventing any change of injection site and leading to discard the injection device still containing the substance to be injected.

The present invention aims to address the above mentioned drawbacks of the prior art and to propose an injection device comprising a needle and a needle shield covering the needle to avoid injuries, the needle shield being locked only after use of the injection device while allowing the user to change the injection site. In other words, the invention aims to propose an injection device comprising a needle and a needle shield lockable in a safety position after injection, while the user can inject a first portion of the substance, change injection site and inject the remaining substance.

### SUMMARY OF THE INVENTION

To this end, a first aspect of the invention is an injection device for injecting a substance into a body, comprising:

a syringe comprising:
a needle to prick the body,
a barrel arranged to contain the substance to be injected,
a stopper arranged in sliding engagement inside the barrel,
a plunger unit coupled to the stopper and moveable from an initial, proximal position to a final, distal position to inject the substance,

a case accommodating and securing the barrel and provided with elastic means and an operating stop,

a needle shield adapted to be moveable under the force of the elastic means from an injection position, into which the needle shield is moveable with regard to the injection needle, to a safety position into which the needle shield permanently covers the injection needle, the needle shield being provided with a flexible leg comprising a shield stop,

wherein the operating stop is configured to prevent the needle shield to move to the safety position by abutting against the shield stop,

wherein the plunger unit comprises a triggering portion configured to deflect the flexible leg of the needle shield when the plunger unit has reached a triggering position during its injection movement from the initial, proximal position to the final, distal position, in order to remove the abutment between the shield stop and the operating stop and to allow the needle shield to reach the safety position under the force of the elastic means.

The flexible leg of the needle shield is thus deflected outside an abutment axis of the operating stop by the trigger portion. Thanks to the trigger unit provided on the plunger unit, the needle shield is thus allowed to move to the safety position only when the plunger unit has reached the triggering position, during its injection movement. This allows a reliable injection position of the needle shield, moving between a retracted and a deployed position as many times as required in order to change injection site. The needle shield can then reach after the injection a reliable safety position that avoids any needle injury.

Preferably, the movement of the needle shield and/or the plunger unit is linear and along the distal-proximal axis i.e. the longitudinal axis of the injection device. This allows for a thin and easy to assemble injection device. More than one flexible leg may be provided on the needle shield, for example, two flexible legs or more, such as four flexible legs. The number of operating stops and the configuration of the triggering portion are preferably adapted to the number of flexible legs. The operating stop of the case may provide a safety stop by limiting a retraction or proximal movement of the needle shield in the safety position.

Advantageously, the injection device further comprises a safety lock arranged for locking the needle shield and/or the shield stop in the safety position of the needle shield. This safety lock maintains the needle shield in a blocked position or strictly limits the movement of the needle shield in the safety position, for example less than a few millimetres. It thus contributes to a safe handling of the injection device after injection.

For example, this safety lock may be a safety stop provided on the case and abutting the shield stop in the safety position of the needle shield. The flexible leg of the needle shield may thus be non-deflected, i.e. in its initial state, in the safety position of the needle shield, to allow this abutment. In other words, the triggering portion of the plunger unit may be configured to deflect the flexible leg only when the needle shield is in a retracted, proximal position.

Alternatively, or in combination, the triggering portion or another portion of the plunger unit may provide for a safety lock in the final, distal position of the plunger unit, for example by a direct contact with the needle shield and/or with the flexible leg, which allows to simplify manufacturing of the injection device.

Advantageously, the safety lock comprises at least one flexible tab provided on the case and the triggering portion is further configured to deflect the flexible tab in order to place it in an abutment position with the shield stop when the plunger unit has reached a safety lock position during its injection movement from the initial, proximal position to the final, distal position. This allows for a thin injection device and a reliable safety position. Alternatively, the flexible tab may be a rotating tab. This safety lock position may be identical to the triggering position of the plunger rod or be another threshold position located after the triggering position in the injection movement.

For example, the triggering portion comprises at least one rigid leg, for example parallel to a longitudinal axis of the injection device, arranged to interact with the flexible leg of the needle shield. Optionally, the triggering portion comprises another rigid leg, that may also be longitudinal, arranged to interact with the flexible tab. For example, the longitudinal rigid legs may have different lengths, i.e. may protrude from the plunger unit at and/or with a different length, in order to interact with the flexible leg of the needle shield and the flexible tab of the case, in the same threshold position of the plunger unit (i.e. the triggering position) or in two different threshold positions (i.e. the triggering position and the safety lock position).

Alternatively, or in combination, the triggering portion may comprise abutment surfaces or protrusions provided on the plunger unit. The triggering portion may comprise two, three or four rigid legs, the needle shield may comprise two, three of four flexible legs and the case may comprise two, three of four operating stops. Each rigid leg is preferably configured to be aligned with an operating stop and a flexible leg on a longitudinal axis or proximal-distal axis of the injection device. Further, two end-of-dose indicators may be provided, each with two flexible arms. The triggering portion may preferably comprise two, or four another legs, preferably aligned with each flexible arms of each end-of-dose indicator on a longitudinal axis or proximal-distal axis of the injection device.

Advantageously, the case comprises at least one longitudinal window allowing a user to view the substance at least when the plunger unit is in the initial, proximal position. This window also allows to see the substance and the movement of the stopper into the barrel during the injection, which allows for a direct control of the injected substance. Preferably, the operating stop, the flexible tab and/or the flexible leg and the shield stop are arranged to be spaced in an area outside of the window area. One, two windows or more may be provided on the case. The window has preferably a length similar or identical to the barrel length.

Preferably, the triggering position of the plunger unit is located in an end portion of the movement or stroke of the plunger unit to the final, distal position or in the final, distal position of the plunger unit. For example, the triggering position is between 85% to 100% of the injection movement of the plunger unit, preferably 90% to 95%. In addition, the safety lock position may be between 85% to 100% or preferably 87 to 100%, 90% to 100% or more preferably 95 to 100% of the injection movement of the plunger unit. Preferably, the triggering position is a position before the safety lock position during the injection movement of the plunger unit.

Advantageously, the injection device further comprises plunger locking means arranged for locking the plunger unit to the case when the plunger unit is in the final, distal position. This provides an additional safety for the injection device after injection, especially to prevent reuse of the injection device. Such plunger locking means are especially useful when the triggering portion or another portion of the plunger unit acts as a safety lock for the needle shield.

Advantageously, the plunger unit comprises a tubular member arranged to accommodate at least part of the case when the plunger unit is in the final, distal position. This tubular member contributes to obtain a “closed” design of the injection device, preventing any reuse and it also enhances the handling of the injection device during pricking and injection.

Advantageously, the case comprises a distal ring defining an abutment surface and the tubular member comprises an end surface, the end surface and the abutment surface being configured to be in contact in the final, distal position of the plunger unit. Preferably, the distal ring and the cover member have the same width or diameter. These features contribute to prevent reuse of the injection device and also provide for a reliable stop of the injection movement of the plunger unit.

For example, a handle may be fixed to the case in order to simplify handling of the injection device and/or to hide the operating stop and/or the flexible tab. For example, the tubular member may be slideable between the case and the handle or onto the handle external surface. The handle may also comprise a window having a size equal or larger than the size of the window of the case.

Advantageously, the plunger locking means comprise an oblique arm provided on one of the tubular member and the case, and a locking recess on the other one of the tubular member and the case, the oblique arm and the locking recess being configured to be engaged in the final, distal position of the plunger unit. Preferably, two oblique arms and two locking recesses are provided. For example, the oblique arm is provided on the tubular member, which simplifies manufacturing of the injection device.

Advantageously, the injection device further comprises:
an end-of-dose indicator accommodated in said case and moveable under the force of the elastic means from a hidden position into which the end-of dose indicator is not visible to a user to a visible position into which at least one portion of the end-of-dose indicator is visible to a user,
hiding means for maintaining the end-of-dose indicator in the hidden position and configured to be unlocked by the triggering portion of the plunger unit, when the plunger unit is in an indicator position during its injection movement from the initial position to the final, distal position, so as to allow automatic movement of the end-of-dose indicator to the visible position.

Such an end-of-dose indicator is useful to provide a clear indication to the user as to when the injection is completed, thus avoiding or limiting the number of injections terminated before the full volume of substance is injected. Since the hiding means are unlocked by the same triggering portion used to trigger the safety position of the needle shield, the design and manufacturing of the injection device may be simplified.

The indicator position may be between 85% to 100% and preferably 95% to 100% or 97% to 100% of the injection movement of the plunger unit. Preferably, the indicator position is the last threshold position of the injection movement, immediately before or on the final, distal position of the plunger unit.

For example, the portion of the end-of-dose indicator is visible in the window and/or in a recess made in the distal ring and/or made in the case.

### BRIEF DESCRIPTION OF THE DRAWINGS

Other features and advantages of the present invention will appear more clearly from the following detailed description of particular non-limiting examples of the invention, illustrated by the appended drawings where:

FIG. 1 represents a perspective view of an injection device according to the present invention, as presented during storage or transportation;

FIG. 2 represents a perspective view of the injection device of FIG. 1, in a ready-to-use state and without the handle;

FIG. 3 represents a cross-section view of the injection device according to FIG. 2;

FIG. 4 represents a perspective view of the injection device of FIG. 3, without the plunger unit;

FIG. 5 represents a perspective cross-section view of the plunger unit of the injection device;

FIG. 6 represents a side view of a distal portion of the injection device of FIG. 2, in a ready-to-use state and a deployed position of the needle shield;

FIG. 7 represents a side view of a distal portion of the injection device of FIG. 2 in a retracted position of the needle shield;

FIG. 8 represents a side view of a distal portion of the injection device of FIG. 2 in the retracted position of the needle shield, wherein the plunger unit has moved distally;

FIG. 9 represents a side view of a distal portion of the injection device of FIG. 2 in the retracted position of the needle shield, wherein the plunger unit is in a final, distal position;

FIG. 10 represents a side view of a distal portion of the injection device of FIG. 2 in a safety position of the needle shield wherein the plunger unit is in a final, distal position;

FIG. 11 represents a cross-section side view of the injection device of FIG. 2 in the safety position wherein the plunger unit is in a final, distal position;

FIG. 12 represents a perspective view of the distal portion of the injection device of FIG. 2 in the ready-to-use position, wherein the needle shield is omitted;

FIG. 13 represents a cross section view of the distal portion of the injection device of FIG. 12, with the end-of-dose indicator shown in the hidden position;

FIG. 14 represents a cross section view of the distal portion of the injection device of FIG. 12 in the final, distal position of the plunger unit, triggering the end of dose indicator;

FIG. 15 represents a cross section view of the distal portion of the injection device of FIG. 12 in the final, distal position of the plunger unit, after the end-of-dose indicator has been triggered;

FIG. 16 represents a cross section view of the distal portion of the injection device of FIG. 12 in the final, distal position of the plunger unit, with the end-of-dose indicator in a visible position.

### DETAILED DESCRIPTION

The present injection system is intended for administration of a substance such as a parenteral drug composition by a medical caregiver or by a patient with a simplified operation and an obvious indication showing when the substance has been fully injected.

As such, in this application, the distal direction must be understood as the direction of injection with reference to the injection system, and the proximal direction is the opposite direction, i.e. the direction toward the hand of the medical caregiver or of the patient. In addition, a drug composition must be understood as all kinds of injectable drug composition adapted for therapeutics, aesthetics, preventive or diagnosis applications.

With reference to FIGS. 1 to 5 is shown an injection device according to a preferable embodiment of the present invention, in a ready-to-use state, i.e. as available to the user after release from a blister packaging.

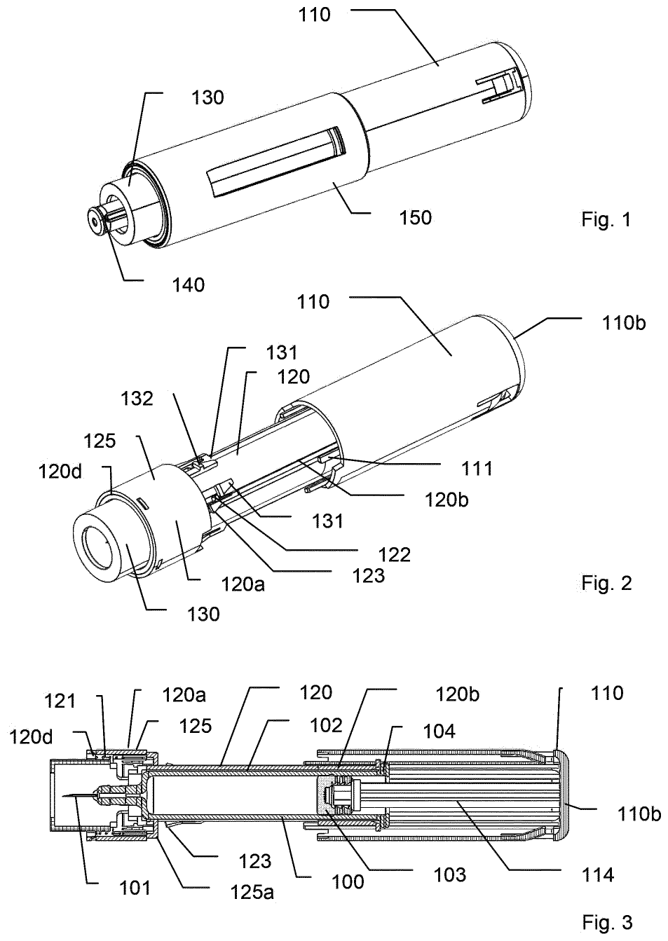

The injection device comprises a case 120, visible in FIGS. 2-4, receiving a syringe 100 having a barrel 102 (only visible in FIG. 3) that contains the substance to be injected. The barrel 102 is provided with a fixed or removable injection needle 101 adapted to prick the patient's body and a stopper 103 in sliding engagement inside the barrel (102), as it is known from the prior art. The barrel 102 further comprises a proximal flange 104 which is held by the case in order for the case to accommodate and secure the barrel 102. The injection needle 101 may be provided with a needle cap 140 covering the injection needle 101 during storage and transportation of the injection device.

The case 120 comprises a distal portion 120a and a proximal portion 120b, the distal portion 120a that may define a distal ring 125 having a width or diameter larger than the proximal portion 120b. The distal ring 125 may comprise an abutment surface 125a facing the proximal direction. The case 120 further comprises elastic means 121, an operating stop 122 and a flexible tab 123. An optional window 124 may give a visual access to the barrel 102 and to the substance it contains.

The case is covered with an optional covering handle 150 (only represented in FIG. 1) intended to cover the case 120 so as to be easily gripped and held by a hand of the user performing the injection. The handle 150 may be fixed or clipped to the case 120 by its distal extremity, i.e. on or close to the distal ring 125. The proximal portion 120b of the case 120 comprises one or two locking recesses 126.

A plunger unit 110, more visible with reference to FIGS. 2, 3 and 5, is adapted to slide with regard to the case 120 and to be coupled to the stopper 103 by the distal extremity 114d of a plunger shaft 114. It comprises an external tubular member 113 adapted to cover at least part of the case 120 and to slide between the case 120 and the handle 150. The plunger unit 110 is moveable under the manual force applied by a user on a pushing surface 110b, from an initial, proximal position (represented in FIGS. 1-3) to a final, distal position (represented in FIG. 11) to inject the substance into the patient's body.

The plunger unit 110 further comprises a triggering portion in the form of two longitudinal rigid legs 111, 112 having different lengths, wherein the longest rigid leg 112 protrudes distally from the tubular member 113 with regard to shortest rigid leg 111. For example, one or several triggering portions may be provided on the plunger unit 110. In addition, the plunger unit 110 comprises one or two oblique arms 115 protruding proximally from the tubular member 113 and intended to cooperate with the locking recesses 126 in the final, distal position of the plunger unit 110. For example, four triggering portions are provided on the plunger unit 110.

A needle shield 130 is accommodated partially in the distal ring 125 of the case 120 and is slidable in view of the case during an injection position into which an injection can be performed (see FIGS. 1-5). In this injection position, the needle shield 130 can move from a deployed position (see FIGS. 1-6) into which the injection needle (101) is covered to a retracted position into which at least part of the injection needle is not covered (see FIG. 7). In addition, the needle shield 130 is arranged to be moveable from the injection position to a safety position, distal from the deployed position and in which the needle shield 130 permanently covers the injection needle 101.

The needle shield 130 is provided with at least one flexible leg 131 extending proximally and comprising a shield stop 132 under the form of a protrusion and a proximal slope 131b. Elastic means 121 are provided in the case 120, for example in the distal ring 125 in order to apply a distal force on the needle shield 130. The elastic means can comprise a cylindrical steel spring abutting a surface provided inside the case 120, such as the distal face of the abutment surface 125a.

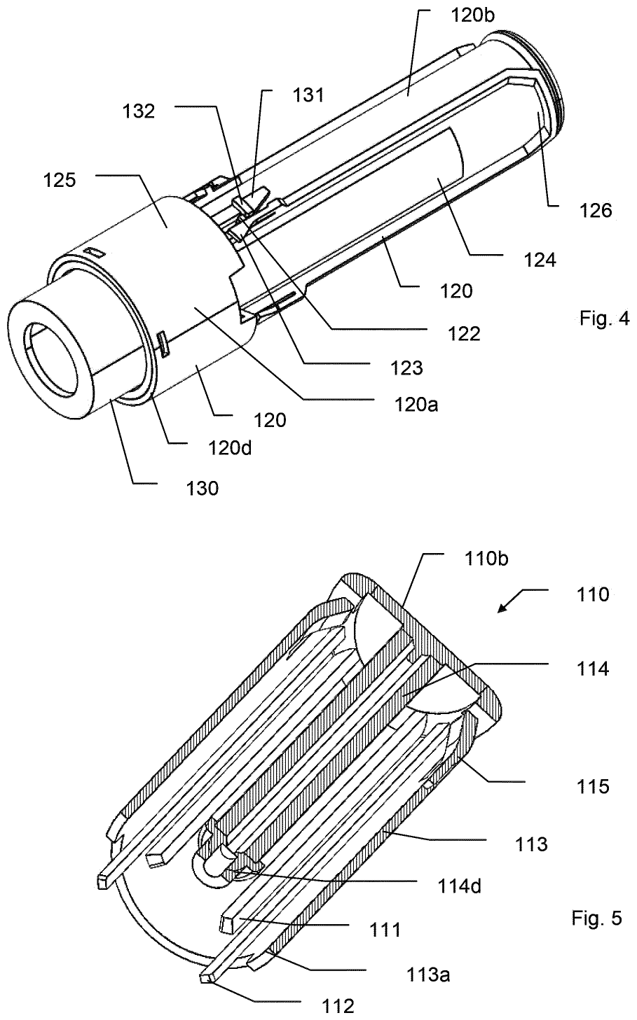

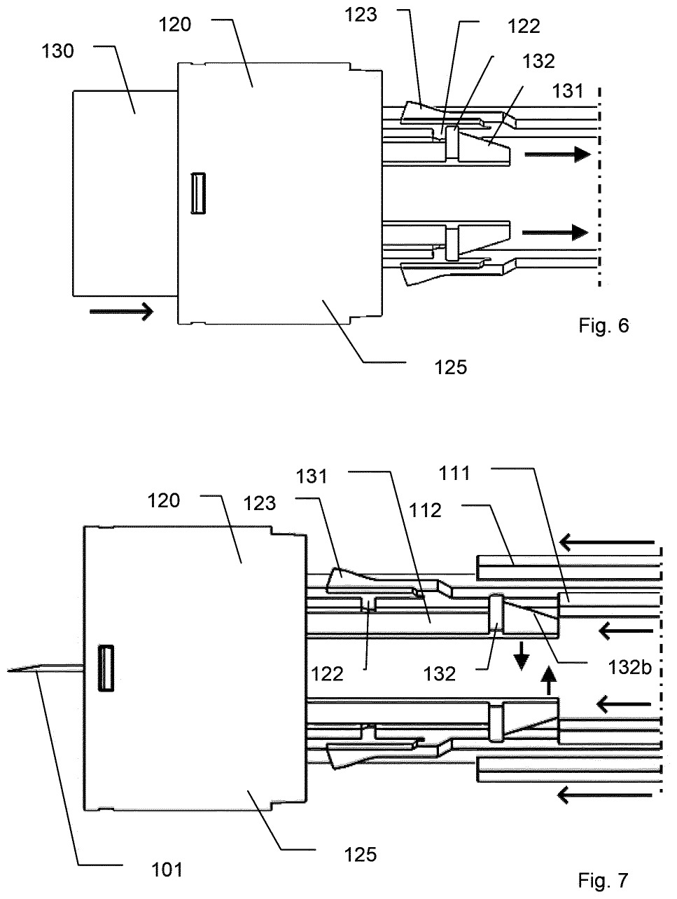

With reference to FIGS. 2 and 4, the case 120 further comprises one operating stop 122 preventing the needle shield 130 to move to the safety position by contacting the shield stop 132 in the deployed position of the needle shield 130, as visible in FIGS. 2-3 and 6. Consequently, in the injection position of the needle shield 130, the shield stop 132 can move from a contact with operating stop 122 to a position proximal to the operating stop 122, when the needle shield 130 moves from the deployed position to the retracted position (see FIGS. 6-7). In the retracted position, the needle shield may be stopped by the elastic means 121. This proximal movement of the needle shield 130 is intended to be performed when the injection device is pressed onto the body with a force greater than the force of the elastic means 121 and as many times as required to complete an injection.

In the figures, two triggering portions, two operating stops and two flexible legs are visible, but one, three of four of them may be provided, in corresponding or aligned positions.

### Operation of the Injection Device

In operation, the injection device may be removed from a blister or packaging and the needle cap 140 may be removed from the injection needle 101. At this moment, the injection needle 101 is covered by the needle shield 130, thus avoiding needle fear.

The injection device is then pressed on the body of a patient and the needle shield 130, in the injection position, moves proximally from the deployed position to the retracted position into which the shield stop 132 of the needle shield 130 is not in contact with the operating stop 122 and the needle shield 130 does not cover at least part of the injection needle 101 (see FIGS. 6-7). The injection needle 101 thus penetrates the patient's body and the pricking is performed.

At this point, it is possible that the patient or the user prefers to change the injection site for medical and/or convenience reasons. This change is permitted by the injection device, thanks to the injection position of the needle shield 130. When the injection device is removed from the patient's body before the injection has been completed, the needle shield 130 is pushed from the retracted position to the deployed position by the elastic means 121 but prevented from reaching the safety position by the operating stop 122. Consequently, the injection needle is covered at any time, especially when not inserted into the patient's body and the same operation of pricking can be reproduced at another injection site of the body.

When the patient or the user is satisfied with the injection site, he/she may press the pushing surface 110b of the plunger unit 110, thus moving the stopper 103 distally and performing the injection of the substance into the body. This distal movement of the plunger unit 110 brings the trigger portion (rigid legs 111 and 112) of the plunger unit 110 closer to the flexible leg 131 of the needle shield 130 and the flexible tab 123 of the case 120, as visible in FIG. 7.

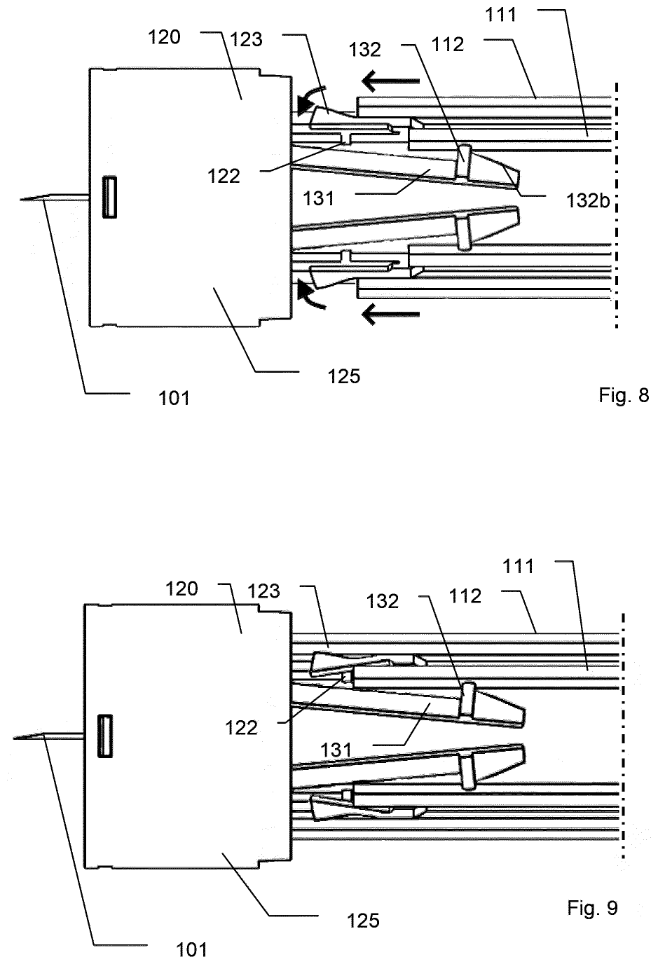

When the plunger unit 110 has reached a first threshold position or triggering position, preferably in an end portion of its injection movement, the shortest rigid leg 111 comes into contact with the proximal extremity and the proximal slope 132b of the flexible leg 131, as visible in FIG. 7. As the plunger unit 110 is moved further in the distal direction to complete the injection, the shortest rigid leg 111 contacts and deflects the flexible leg 131, thanks to its proximal slope 131b, according to the arrows represented in FIG. 7 and as visible in FIG. 8.

In a safety lock position of the plunger unit 110, which corresponds to the first threshold position or a second threshold position in the injection movement of the plunger unit 110, the longest rigid leg 112 comes into contact with the flexible tab 123. The flexible tab 123 is then moved into a space distal from the operating stop 122, according to the circular arrows of FIG. 8 and as represented in FIG. 9. The plunger unit 110 still moves under the user's force in the distal direction and the shortest rigid leg 111 moves into a space proximal from the operating stop 122 up to abutting the operating stop 122. Alternatively or in combination, the plunger unit 110 may move until the stopper 103 abuts the end of the barrel 102. The flexible leg 131 is maintained in a deflected position and the shortest rigid leg 111 occupies the space previously occupied by the flexible leg 131, as represented in FIG. 9. At the same time, the longest rigid leg 112 moves toward the distal ring 125 and maintains the flexible tab 123 in a deflected position distally from the operating stop 122.

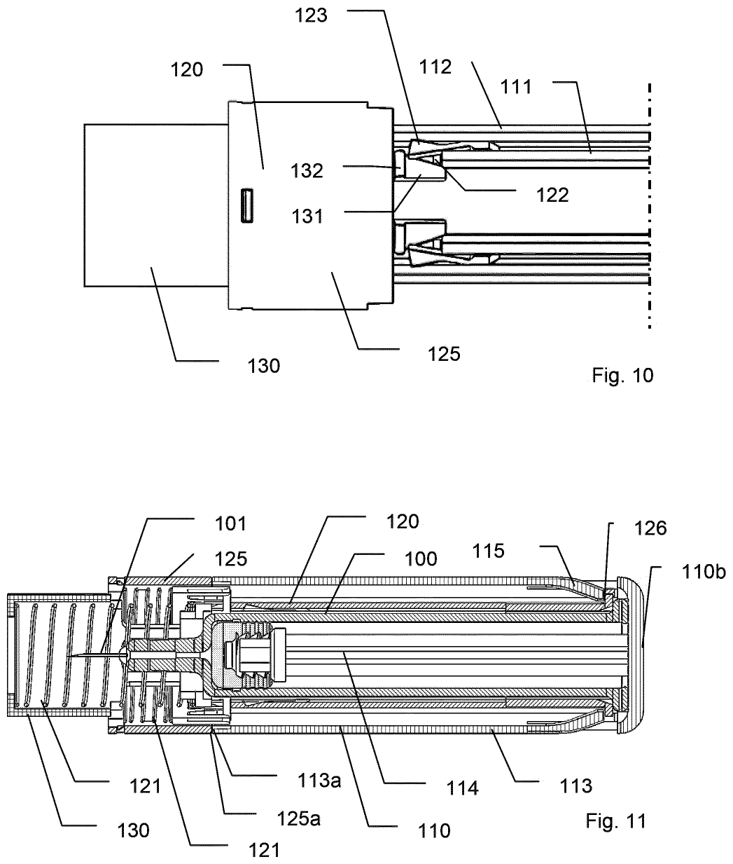

At the end of the injection, when the plunger unit 110 reaches its final, distal position and all the substance contained in the barrel 102 has been transferred to the body through the injection needle 101, the injection device is as represented in FIGS. 9 and 10. In this position, the shortest rigid leg 111 may be in abutment on the operating stop 122 and/or the stopper 103 may abut the distal end of the barrel 103.

The injection device is then removed from the injection site and the needle shield 130 is pushed distally by the elastic means 121. However, the flexible leg 131 is still deflected by the shortest rigid leg 111 and the shield stop 132 cannot contact the operating stop 122 anymore. When the needle shield moves distally, the shield stop 132 reached the space located distally from the operating stop 122, into which it comes to a stop on the abutment surface 125a of the distal ring 125. Further any proximal movement of the needle shield is blocked by a contact between the shield stop 132 and the flexible tab 123, maintained in the flexible position by the longest rigid leg 112, as visible in FIG. 10.

In FIG. 10, the needle shield is in the safety position covering the whole injection needle 101, so as to avoid any needle pricking, and is prevented from moving proximally by the abutment between the shield stop 132 of the flexible leg 131 and the flexible tab 123 of the case 120. The shortest rigid leg 111 and the longest rigid leg 112 thus act as a triggering portion in order to trigger the safety position of the needle shield 130, by allowing the flexible leg 131 to escape the operating stop 122 and the flexible tab 123 to reach an abutment position into which it can act as a safety lock.

As represented in FIG. 11, in the final distal position of the plunger unit 110, the oblique arm 115 of the plunger unit 110 is engaged with the locking recess 126 of the case 120, and the end surface 113a of the tubular member 113 contacts the abutment surface 125a of the distal ring 125. Consequently, the plunger unit 110 is locked with regard to the case 120, thus preventing any reuse of the injection device and allowing for a safe disposal of the used injection device.

### End-of-Dose Indicator

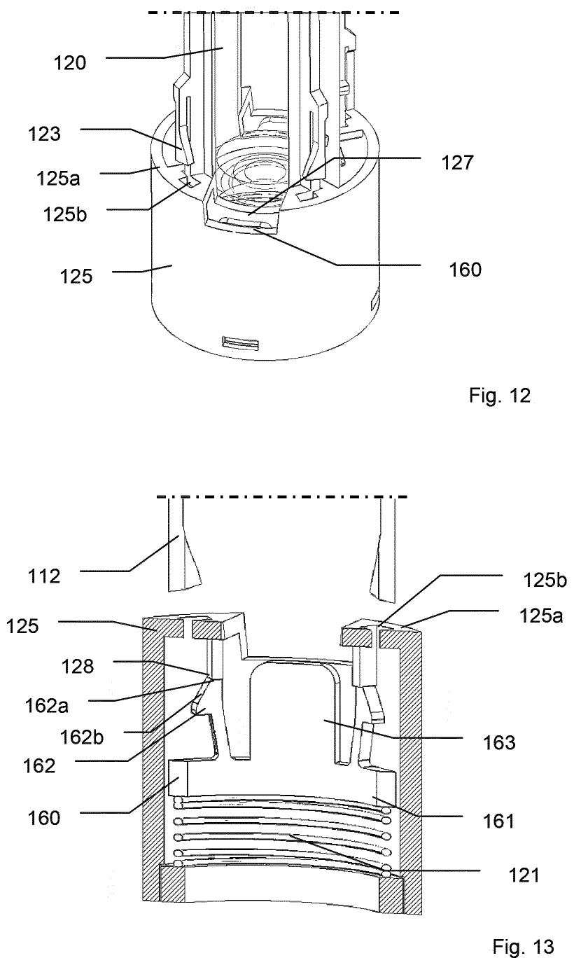

Optionally, the injection device according to the present invention may comprise an end-of-dose indicator 160, as described with reference to FIGS. 12-16. Please note that neither the plunger shaft 114 nor the needle shield 130 are represented in FIGS. 12-16 for clarity reasons.

The end-of-dose indicator 160 may be located in the distal ring 125 of the case 120 and the distal ring 125 may comprise a triggering opening 125b provided in the abutment surface 125a and aligned axially to receive the longest rigid leg 112 of the plunger unit 110. In addition, the case 120 may also comprise an indicator opening 127 configured to render visible at least part of the end-of-dose indicator 160 after the injection has been completed.

With reference to FIG. 13, the end-of-dose indicator 160 comprises a base 161 in contact with the elastic means 121 in order to receive a proximal force from the elastic means 121. The end-of-dose indicator 160 further comprises at least one and preferably four flexible arms 162 protruding from the base 161 in the proximal direction and each comprising an extremity provided with an abutment portion 162a and a slope 162b.

The end-of-dose indicator 160 is moveable from a hidden position into which the end-of-dose indicator 160 is hidden from a user in the case 120, to a visible position into which at least one portion of the end-of-dose indicator such as a tab 163 is visible to a user and/or protrude outside the case 120 and/or through the indicator opening 127. For example, the tab 127 may be a portion of the end-of-dose indicator with a visible color and/or a visible design, such as a red rectangle or a rectangle with a red dot.

The case 120 comprises an indicator stop 128, such as one or two rigid arms pointing in the distal direction, for example inside the distal ring 125. In the ready-to-use state represented in FIGS. 12-13, the abutment portion 162a of each flexible arm 162 is pushed against the indicator stop 128 of the case 120 by the elastic means 121. Consequently, the end-of-dose indicator 160 is blocked in the hidden position by these hiding means. The elastic means may be the same spring as used for the needle shield or another concentric spring abutting an internal abutment surface of the distal ring 125.

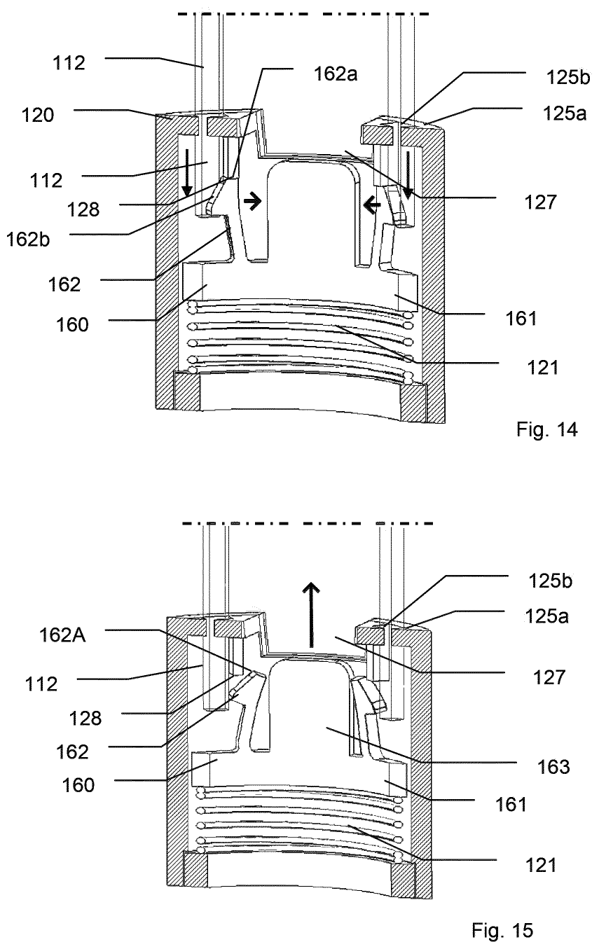

When the plunger unit 110 moves from the initial, proximal position to the final distal position, the triggering portion such as the longest rigid leg 112 penetrates in the distal ring 125 through the triggering opening 125b, as visible in FIG. 14, in which the longest rigid leg 112 is shown as transparent. When the plunger unit has reached an indicator position, which may be the second threshold position or a third threshold position, the longest rigid leg 112 comes in contact with the slope 162b of the flexible arm 162 and the flexible arm is thus deflected toward the tab 163. The contact between the abutment portion 162a and the indicator stop 128 is thus removed and these hiding means are deactivated or unlocked, as represented in FIG. 15.

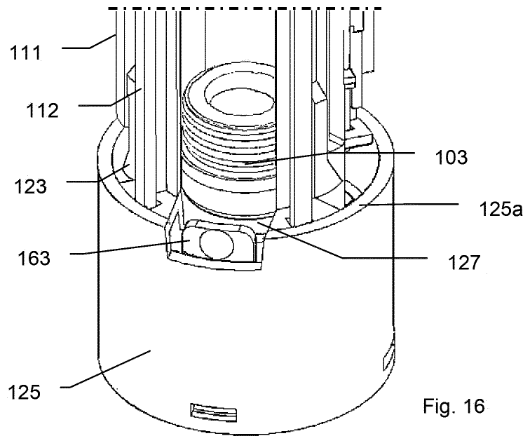

With reference to FIG. 15, the end-of-dose indicator 160 is free to move to the visible position and the tab 163 may pop up into the indicator opening 127, as visible in FIG. 16 by the user. Thanks to the quick trigger of the hiding means and the immediate movement of the end-of-dose indicator 160 in the visible position, the user and/or the patient is informed that the substance has been fully injected without any ambiguity.

### OTHER FEATURES AND ALTERNATIVES

It is, of course, understood that obvious improvements and/or modifications for one skilled in the art may be implemented, still being under the scope of the invention as it is defined by the appended claims.

For example, the elastic means are not limited to one or two cylindrical springs but may also comprise any form of spring or elastic means. The external surface of the tubular member may have an ergonomic relief and may comprise overmoulded surfaces in smooth material. The shield stop 132 can have another design than a protrusion such as for example a cavity or a groove

The materials intended to build such an injection device are polypropylene, polycarbonate and/or acrylonitrile-butadiene-styrene. The barrel may comprise glass or plastic. Colour may be provided on various parts of the injection device, in particular on the tab of the end-of-dose indicator. The window may be provided with graduation or indicator.

1. An injection device for injecting a substance into a body, comprising:
a syringe comprising:
an injection needle to prick the body;
a barrel arranged to contain the substance to be injected;
a stopper arranged in sliding engagement inside the barrel; and
a plunger unit coupled to the stopper and moveable from an initial, proximal position to a final, distal position to inject the substance;
a case accommodating and securing the barrel and provided with elastic means and an operating stop; and
a needle shield adapted to be moveable under the force of the elastic means from an injection position into which the needle shield is moveable with regard to the injection needle to a safety position into which the needle shield permanently covers the injection needle, the needle shield being provided with a flexible leg comprising a shield stop,
wherein the operating stop is configured to prevent the needle shield to move to the safety position by abutting against the shield stop,
wherein the plunger unit comprises a triggering portion configured to deflect the flexible leg of the needle shield when the plunger unit has reached a triggering position during its injection movement from the initial, proximal position to the final, distal position, in order to remove the abutment between the shield stop and the operating stop and to allow the needle shield to reach the safety position under the force of the elastic means.

2. The injection device according to claim 1, wherein the injection device further comprises a safety lock arranged for locking the needle shield and/or the shield stop in the safety position of the needle shield.

3. The injection device according to claim 2, wherein the safety lock comprises at least one flexible tab provided on the case and the triggering portion is further configured to deflect the flexible tab in order to place it in an abutment position with the shield stop when the plunger unit has reached a safety lock position during its injection movement from the initial, proximal position to the final, distal position.

4. The injection device according to claim 1, wherein the case comprises at least one longitudinal window allowing a user to view the substance at least when the plunger unit is in the initial, proximal position.

5. The injection device according to claim 1, wherein the triggering position of the plunger unit is located in an end portion of the movement of the plunger unit to the final, distal position or in the final, distal position of the plunger unit.

6. The injection device according to claim 1, wherein the injection device further comprises plunger locking means arranged for locking the plunger unit to the case when the plunger unit is in the final, distal position.

7. The injection device according to claim 1, wherein the plunger unit comprises a tubular member arranged to accommodate at least part of the case when the plunger unit is in the final, distal position.

8. The injection device according to claim 7, wherein the case comprises a distal ring defining an abutment surface and the tubular member comprises an end surface, the end surface and the abutment surface being configured to be in contact in the final, distal position of the plunger unit.

9. The injection device according to claim 6, wherein the plunger locking means comprises an oblique arm provided on one of tubular member and the case, and a locking recess on the other one of tubular member and the case, the oblique arm and the locking recess being configured to be engaged in the final, distal position of the plunger unit.

10. The injection device according to claim 1 further comprising:
an end-of-dose indicator accommodated in said case and moveable under the force of the elastic means from a hidden position into which the end-of dose indicator, is not visible to a user to a visible position into which at least one portion of the end-of-dose indicator is visible to a user; and
hiding means for maintaining the end-of-dose indicator in the hidden position and configured to be unlocked by the triggering portion of the plunger unit, when the plunger unit has reached an indicator position during its injection movement from the initial, proximal position to the final, distal position, so as to allow automatic movement of the end-of-dose indicator to the visible position.

11. The injection device according to claim 1, wherein a safety lock position of the plunger unit is located on or after the triggering position during the movement of the plunger unit from the initial, proximal position to the final, distal position.
